(19) United States
(12) Patent Application Publication
(10) Pub. No.: US 2009/0197260 A1
(43) Pub. Date: Aug. 6, 2009

(54) METHOD FOR PREDICTING THE VIABILITY AND VITALITY OF BACTERIA USABLE IN STRESSING ENVIRONMENT

(75) Inventors: Jean Guzzo, Fontaine Les Dijon (FR); Nicolas Desroche, Dijon (FR); Raphelle Tourdot-Marechal, Dijon (FR)

Correspondence Address: YOUNG & THOMPSON, 209 Madison Street, Suite 500, ALEXANDRIA, VA, 22314, US

(73) Assignee: UNIVERSITE DE BOURGOGNE, DIJON (FR)

(21) Appl. No.: 12/095,812

(22) PCT Filed: Dec. 1, 2006
(86) PCT No.: PCT/FR06/02633

§ 371 Date: Dec. 8, 2008

(30) Foreign Application Priority Data
Dec. 2, 2005 (FR) 0512292

(51) Int. Cl. C12Q 1/68

(52) U.S. Cl. 435/6

(57) ABSTRACT

A method for predicting viability, vitality and stability of bacteria, which are usable in stressing environments, produced in the form of bacterial preparations and packed in a frozen or lyophilised form, includes pre-suspending the bacterial preparations in non-stressing aqueous nutrient medium, carrying out a molecular measurement of at least one type of marker gene whose stress state is representative for each considered bacterium species, measuring intracellular pH and in comparing the measured values with reference and/or reference standard values. The use of the inventive method for controlling quality of stress adaptation of bacterial preparations and for selecting such preparations is also disclosed. The invention in particular relates to malolactic and probiotic ferments.

The present invention relates to a method for predicting the viability, vitality and stability of bacteria, which are usable in stressful environments and the use of said method for controlling the quality of stress adaptation of bacterial preparations constituted from said bacteria and/or for selecting bacteria entering in the composition of such preparations.

In the field of vinification, yeast ferments are used for alcoholic fermentation and bacterial ferments are used for malolactic fermentation.

To this end, preparations of bacterial ferments have been developed which enable activation of malolactic fermentation at the suitable time of the vinification process, via inoculation of the ferments in the wine. Said inoculation, or seeding, may be produced directly or after reactivation of the ferments. Oenococcus oeni is the bacterial species the most often used and the best suited for producing said fermentation.

The manufacturers of said bacterial preparations and the users thereof are confronted with the problem of the survival of the bacteria during inoculation in the wine: indeed, the physical-chemical conditions of the wine are extremely unfavourable to the implantation of said lactic ferments and therefore cause high mortality of the bacteria. In order to reduce said problem and optimise the use of said malolactic ferments, the manufacturers of bacteria have developed pre-acclimatization protocols for the bacteria. Bacterial cultures are subjected to a series of physical-chemical stresses forcing the bacteria to adapt the cellular structure thereof and the metabolism thereof, enabling strains to be obtained, that are capable of surviving after seeding in the wine.

More generally, bacterial ferments, and in particular lactic ferments, are subjected to unfavourable conditions (acidity, nutritional deficiency, oxidative stress, etc.) during the implementation thereof. The producers of said bacterial preparations are therefore confronted with the problem of guaranteeing a survival, vitality and stability of said preparations during the storage thereof or during the use thereof in fermentary methods or even, in the case of probiotics, during the ingestion thereof and notably when passing through the stomach. Probiotics means food supplements made from live bacteria which exert a beneficial effect by improving the equilibrium of the intestinal flora; said probiotic ferments contain lactobacilli, bifidobacteria and streptococci.

Currently, the difficulty for said producers of bacterial preparations is therefore to obtain a reliable measurement of the level of stress adaptation of bacterial preparations, pre-acclimatized or not, and the ability thereof to survive after inoculation in the media; it is also important for same to validate the quality of the products obtained, after freezing or lyophilisation, which, enables same to propose bacteria which perform the best in commercial preparations.

In the case of malolactic ferments, industrialists currently use micro-vinification experiments which are expensive, long (a plurality of weeks) and labour costly. In addition, they are far removed from real conditions of use of ferments, due to the low volumes considered and the difficulty of using in said experiments a wine which represents the wines of the end users of said ferments.

Other tests have also been developed, based on the influence of bacteria on the physical parameters of the environment thereof, such as the concentration of certain metabolites that said bacteria consume or produce.

The conventional technique of cell count is also used, which consists of taking regular samples of the stressful environment wherein, the bacteria have been implanted, then culturing said samples and counting the bacteria after a predetermined incubation time. Now if said technique makes it possible to observe the growth of the bacterial population subjected to stress, it only partially provides information on the physiological state of the bacteria: indeed, it is known that bacteria may multiple in a culture medium without being fully functional and conversely, bacteria adapted to stress conditions may not be able to multiply in a culture medium. Said technique therefore does not enable the capacity of the bacteria to survive in stressful environment to be controlled reliably and reproducibly, after the industrial preparation thereof.

None of said tests is currently truly satisfactory: on one hand, they are tedious and demand a long execution time, and on the other hand, the degree of accuracy thereof does not give full satisfaction in terms of quality control tools.

The aim of the present invention is therefore to circumvent the abovementioned difficulties by proposing a reliable, reproducible, accurate and fast method which, on one hand, makes it possible to estimate and validate the level of stress adaptation of bacterial preparations produced industrially and which are usable in environments hostile to the survival of said bacteria and, on the other hand, to select the bacterial strains that constitute such preparations.

In relation thereto, the subject matter of the present invention is a method for predicting the viability, vitality and stability of bacteria, which are usable in stressful environments, bacteria which are produced in the form of bacterial preparations and packed in a frozen or lyophilised form, noteworthy in that said method consists of:
a step for pre-suspending said bacterial preparations in a non-stressful aqueous nutrient medium;
carrying out a molecular measurement of the level of expression of at least one marker gene representing the stress state for each bacterial species considered;
measuring the intracellular pH of the bacteria based on a pH-sensitive fluorescent probe;
a step for comparing the values measured with those of references and/or reference standards.

Aqueous nutrient medium means a medium containing substances that are useful for the normal activity of the bacteria and the survival thereof, without in as much constituting a growth medium with would enable said bacteria to multiply; furthermore, said medium is non-stressful, i.e. it constitutes a neutral environment on the physical-chemical level, which enables the bacteria to have a normal energetic activity. For example, such a medium is peptone water (NaCl at 9 g·L⁻¹ and peptone at 1 g·L⁻¹), which is conventionally used in microbiology for dilution operations.

The last step of the method according to the invention makes it possible to accurately define the vitality, viability and stability features of the bacteria considered when they will later be placed in a stressful environment.

The subject matter of the present invention is also the use of said method for controlling the stress adaptation of bacterial ferments. Said ferments may be obtained via pre-acclimatization methods or via pre-selections of bacterial strains. The adaptation to acid stress is more particularly concerned. One of the particularly preferred uses concerns controlling malolactic ferments which are useable for seeding wine.

According to a first aspect of the invention, a molecular measurement is carried out which is based on measuring the level of expression of at least one marker gene representing the stress state of the bacterial species considered.

According to a second aspect of the invention, the internal pH is measured, i.e. a measurement of the physiological state which translates the viability and vitality of bacteria and which is linked to the membrane integrity of bacteria.

The characterisation of the physiological stability over time of said bacteria is carried out by repeating both types of measurements over a period of time corresponding to the time for storing and/or using bacteria.

It is well understood that the combination of said two parameters makes it possible to reliably interpret the quality of survival and activity of the bacteria after inoculation: via quantification, of the gene expression, the adaptation to stress conditions of the bacteria is verified, and via characterisation of the physiological state, it is ensured that said bacteria are viable and have a satisfactory level, of vitality, the correlation of said data resulting in complete and meaningful information on the adaptation of bacteria.

The values measured are compared with those of references and/or standards in order to define the degree of gene expression and the nature of the physiological state in order to characterise the bacteria tested. The references will generally be the same bacterial strains, that have not been subjected to stressful environment pre-acclimatization. Other advantages and features of the invention will emerge better from the detailed description which will follow of the method according to the invention.

According to an essential feature of the invention, the method implements a measurement of the level of expression of at least one marker gene representing the stress state of the bacteria.

Said level of expression is quantified by the inverse transcription technique coupled with the real-time quantitative PCR (RT-qPCR).

Preferably, the measurement of the response to the stress will concern the expression of a plurality of marker genes, which increases the reliability of the measurement. Advantageously, genes which are expressed at various growth stages of the bacteria will be chosen. Preferably, genes will be chosen for which the ratio of the levels of expression between stress adapted population and non-adapted population is at least a factor 2.

Thus, in the case of malolactic ferments and the Oenococcus oeni bacterium, the expression of the hsp18 gene will be at least that measured within the context of the method. Said gene encodes the small Hsp Lo18 protein chaperone.

Preferably, the measurement of the expression of at least one of the genes taken from clpX, trxA or cfa, which are involved in the adaptation of Oenococcus oeni to stress, will be added. The expression of hsp18 is significantly induced by multiple stresses, also during the stationary phase. Conversely, ClpX is distinguished as a marker of the lag phase whereas trxA and cfa are stress markers expressed during all of the phases.

The Person skilled in the Art will refer to genetic databases publishing the Oenococcus oeni genome to determine the nucleic probe sequences which will be used during the RT-qPCR. Advantageously, reference will be made to the BELTRAMO et al. publication, Real-time PCR for characterizing the stress response of Oenococcus oeni in a wine-like medium. Research in microbiology, vol. 157, no. 3, 02 September 2005, pages 267-274, which presents possible sequences of nucleic primers.

Reference will also be made to the following publications, the contents of which form an integral part of the description of the present patent, for the features of said marker genes for Oenococcus oeni:
Jobin, M. P., Delmas, F., Garmyn, D., Diviès C. and Guzzo, J. Molecular characterization of the gene encoding a 18-kDa small heat shock protein associated with the membrane of Leuconostos oenos. Appl. Env. Microbiol. 63: 6309-614 (1997).
Jobin, M. P., Garmyn, D., Diviès, C. and Guzzo, J. Expression of the Oenococcus oeni trxA gene is induced by hydrogen peroxide and heat shock. Microbiology. 145: 1245-1251 (1999).
Jobin, M. P., Garmyn, D., Diviès, C. and Guzzo, J. The Oenococcus oeni clpX homologue is a heat shock gene preferentially expressed in exponential growth phase—J. Bacteriol. 181: 6634-6641 (1999).

In the case of Lactobacillus plantarum, which is a probiotic, advantageously, at least the measurement of the expression of the hsp18.55 gene could be used. Preferably, for better relevance of the results, the measurement of the expression of at least one of the genes taken from hsp19.5, groEL or cfa will be added.

Of course, in the case of other bacterial preparations, and in particular probiotics, the Person skilled in the Art will choose the most suitable gene markers. Preferably, encoding genes will be targeted for proteins of low molecular mass induced during stress and for which the ratio of the level of expression between stress adapted population and non-adapted population is at least a factor 2. Indeed, such genes, which correspond for example to hsp18 with O. oeni or to hsp18.55 or hsp19.5 with L. plantarum, are also present with bacterial types usually used such as probiotics (Lactobacilli, bifidobacteria).

The protocol of the molecular measurement is based on the conventional application of the RT-qPCR technique and is broken down according to the following main steps:
a first step for suspending lyophilised or frozen bacterial preparations, for example via rehydration for fifteen minutes at 30° C. in peptone water (NaCl at 9 g·L⁻¹ and peptone at 1 g·L⁻¹) if the preparations are lyophilised;
extraction of the RNA and dosages of RNA via spectrophotometric measurement at 260/280 nm;
treatment with DNase and verification of the efficacy of the treatment via PCR;
reverse transcription;
quantitative PCR with specific initiations of the targeted genes and use of an internal representative for standardisation of the results.

A calibration and/or comparison is then used with the representative strains to qualify the stress adaptation of the bacteria according to the gene adaptation of the bacteria according to the gene expression measured.

In the case of Oenococcus oeni strains, measurement ratios of the transcript levels are thus obtained between those of the acclimatized cells and those of the same non-acclimatized strains of 5 for hsp18, of 2.25 for clpX, of 5.5 for trxA, and of 4 for cfa. The strains have been acclimatized by subjecting same to an industrial pre-acclimatization process applied by the manufacturers of malolactic ferment.

In the case of Lactobacillus plantarum strains, the ratio of the transcript levels between the cells acclimatized by cultures at acid pH and cells not acclimatized is 2.1 for hsp18.55, 3.4 for hsp19.5, 2.8 for groEL, and 2.6 for cfa.

According to another essential feature of the invention, the methods implement a measurement of the physiological state which translates both the viability and vitality of the bacteria, which is based on a measurement of the intracellular pH (pHi) of the bacteria. Indeed, not only does said technique provide us with information on the membrane integrity but it is also significant of the survival capacity in acid medium, a fundamental parameter notably in the case of lactic ferments.

Indeed, the bacteria which have adapted well to stress conditions have a pHi significantly different from the non-adapted representatives. Therefore, it is not possible to determine, for each bacterial species and for a given medium, the threshold values and/or the ranges of pHi values with which the global physiological state of the bacteria is characterized. Thus, in the case of non-acclimatized and acclimatized Oenococcus oeni strains, it is determined that for a pH value of the external medium of 3.5, that the pHi of the non-acclimatized, cells is 5.21±0.15 whereas that of the acclimatized cells is 4.99±0.05.

In the case of Lactobacillus plantarum, the pHi for an external pH at 3.5 of the non-acclimatized cells is 5.41+0.06 whereas that of the acclimatized cells is 5.02±0.06.

The protocol implemented may be based on an intracellular pH measurement via a pH-sensitive fluorescent probe, for example carboxy fluoresceine diacetate (CFDA). Said protocol derives from the technique described by Breeuwer et al. (Applied and Environmental Microbiology, January 1996).

The protocol applied for Oenococcus oeni is broken down according to the following steps:
a first step for re-suspending lyophilised or frozen bacterial preparations, in a non-stressful nutrient medium; for example, for Oenococcus oeni lyophilised preparations are re-hydrated for 15 minutes at 30° C. in peptone water (NaCl: 9 g·L⁻¹ and peptone: 1 g·L⁻¹);
washing of the cells in a 100 mM Potassium Phosphate buffer, pH 5.3;
re-suspending the cells in the 100 mM Potassium Phosphate buffer, pH 8.0 with the CDCFDase probe at 1 μM and 30 minutes incubation at 30° C.; during this step the probe is loaded and conjugated;
centrifugation and re-suspension of the cells in the 100 mM Potassium Phosphate buffer, pH 5.3 with glucose at 20 μM and 30 minutes incubation at 30° C.; elimination of the non-conjugated probe is carried out;
centrifugation and re-suspension of the cells in the 100 mM Potassium Phosphate buffer, pH 5.3, in order to carry out elimination of the non-conjugated probe present in the supernatant;
centrifugation and re-suspension in 2 mL of 1.00 mM Citrate/Phosphate buffer at various pHs. Addition of 1 μL of nigericin and 1 μL of valinomycin for calibrating the probe; the aim of this step is the permeabilisation of the membrane to protons;
reading of the fluorescence emitted at 531 nm after excitation to 440 nm (emission insensitive to the pH) and to 512 nm (emission sensitive to the pH) on the non-filtered suspension (total fluorescence) and on the filtered suspension on a 0.22 μm filter (extracellular fluorescence);
calculation of the ratio=(Emission after Ex: 512 nm non-filtered—Emission after Ex: 512 nm filtered)/(Emission after Ex: 490 nm non-filtered—Emission after Ex: 490 nm filtered);
use of the calibration curve: intracellular pH=f (512/440 nm ratio) to determine the intracellular pH;

In the case of Lactobacillus plantarum, the same measurement protocol was applied with the following modifications: ph of the 1.00 mM Potassium phosphate buffer at pH 6.0 and 35° C. incubation temperature instead of 30° C.

The Person skilled in the Art will adapt the protocol to the specificities of each bacterial strain according to the optimum growth pH and temperature.

The compositions of the buffer solutions are to be adapted by the Person skilled in the Art for each type of bacterial strain tested, in order to optimise the measurement.

The prediction method was validated for Oenococcus oeni by comparing with the non-acclimatized preparation parameters and by carrying out micro-vinification experiments enabling the measurements made to be correlated with the survival after implantation of the bacteria in the wine. Thus, the acclimatized preparations have a high level of expression of marker genes and an intracellular pH significantly different in relation to the references, non-acclimatized. The micro-vinification experiments have highlighted a growth of the pre-acclimatized bacterial population, associated to a reduction in the malic acid concentration, whereas the non-acclimatized populations survived with great difficultly and had no incidence on the level of malic acid.

In the case of the Lactobacillus plantarum probiotic, the method was validated via correlation with the survival of acclimatized and non-acclimatized bacteria which were inoculated in a non-nutrient medium at acid pH of 2.9, simulating the acid conditions of the gastric fluid of the stomach.

The advantage of the use of said, prediction method as a quality control tool is well understood, which makes it possible to very rapidly obtain an indication of the degree of stress adaptation, and in particular acid stress, of bacterial preparations, irrespective of whether they are obtained, by selection or by pre-acclimatization.

It will be noted that the prediction method used is particularly robust on the statistical level, the measurements made and the results obtained prove to be accurate and reproducible.

Said method further enables industrialists to adjust the pre-acclimatization parameters thereof to be much more effectively adjusted and the pre-acclimatization processes thereof to be more easily optimised. Indeed, the gene expression measurement can be produced within 48 hours as of receipt of the bacterial culture, whereas the intracellular pH measurement requires 4 to 5 hours.

The quality control of the stability of said bacterial preparations during the storage and/or use thereof, whilst repeating over time the measurements of the prediction method, is therefore also much easier and faster in relation to known techniques.

The same applies to the use of the method according to the invention for selecting bacterial strains, in particular probiotic ferments.

The method, according to the invention will in particular have a use for controlling the quality and/or selection of bacterial preparations in the fields of human and animal nutrition (probiotics), agri-food (fermentation processes, in particular wine, beer, maize, soya bean and in curing and pork products), agriculture (bacterial inoculi) and the environment (composting, fight against pathogenic bacteria and hygenisation of effluents).

The method will also be used according to the invention to optimise the methods for producing bacterial ferments, which are useable in stressful environments.

Finally, it is obvious that the method according to the invention may be applied without difficulty and without excessive effort for the Person skilled in the Art to other bacterial preparations confronted during the use thereof with stressful and/or hostile environments, the gene markers of course being suitable for the species considered and the threshold values of the pHi being determinable without particular difficulties.

1. A method for predicting the viability, vitality and stability of bacteria, which are usable in stressful environments, bacteria which are produced in the form of bacterial preparations and packed in a frozen or lyophilised form, characterized in that said method comprises:
a step for pre-suspending said bacterial preparations in a non-stressful aqueous nutrient medium;
carrying out a molecular measurement of the level of expression of at least one marker gene representing the stress state for each bacterial species considered;
measuring the intracellular pH of the bacteria based on a pH-sensitive fluorescent probe;
a step for comparing the values measured with those of references and/or reference standards.

2. A method according to claim 1 characterized in that the bacterium is Oenococcus oeni and in that the molecular measurement concerns the expression of the hsp18 gene.

3. A method according to claim 2 characterized in that the molecular measurement further concerns the expression of at least one gene taken from: clpX, trxA, cfa.

4. A method according to claim 1 characterized in that the bacterium is Lactobacillus plantarum and in that the molecular measurement concerns the expression of the hsp18.55 gene.

5. A method according to claim 4 characterized in that the molecular measurement further concerns the expression of at least one gene taken from: hps19.5, groEL, cfa.

6. A method for controlling the quality of stress adaptation of probiotic ferments, comprising subjecting bacteria to the method according to claim 1, and selecting a bacteria to control the quality of stress adaptation of probiotic ferments.

7. A method for controlling the quality of stress adaptation of malolactic ferments which are useable for seeding wine, comprising subjecting bacteria to the method according to claim 1, and selecting a bacteria to control the quality of stress adaptation of malolactic ferments which are usable for seeding wine.

8. A method for selecting bacterial strains used for producing bacterial preparations confronted with stressful environments under culture conditions, comprising subjecting bacteria to the method according to claim 1, and selecting a bacteria for producing bacterial preparations confronted with stressful environments under culture conditions.
